(19) United States
(12) Patent Application Publication
(10) Pub. No.: US 2016/0106655 A1
(43) Pub. Date: Apr. 21, 2016

(54) SKIN CLEANSING COMPOSITION

(71) Applicant: KAO CORPORATION, Tokyo (JP)

(72) Inventors: Kouhei YAMADA, Sumida-ku (JP); Atsushi TOMOKUNI, Sumida-ku (JP)

(73) Assignee: KAO CORPORATION, Chuo-ku, Tokyo (JP)

(21) Appl. No.: 14/787,174

(22) PCT Filed: Apr. 28, 2014
(86) PCT No.: PCT/JP2014/061855

§ 371 Date: Oct. 26, 2015

(30) Foreign Application Priority Data
Apr. 30, 2013 (JP) 2013-096091

(51) Int. Cl. A61K 8/73; A61K 8/81; A61K 8/06; A61K 8/31; A61Q 1/14; A61K 8/33

(52) U.S. Cl. CPC A61K 8/731; A61Q 1/14; A61K 8/33; A61K 8/06; A61K 8/31; A61K 8/8147; A61K 8/8152

(57) ABSTRACT

Provided is a skin cleansing composition comprising the following components (A), (B), and (C): (A) from 0.5 to 40 mass % of an ether oil in a liquid state at 25° C., (B) from 0.01 to 5 mass % of a nonionic water-soluble polymer comprising cellulose as a constitutional unit selected from the group consisting of methylcellulose, ethylcellulose, hydroxyethylcellulose, hydroxypropylcellulose, and hydroxypropylmethylcellulose, and (C) water, and 1 mass % or less of a nonionic surfactant, wherein the mass ratio of the component (B) to the component (A), (B)/(A) is from 0.001 to 5.

### FIELD OF THE INVENTION

The present invention relates to a skin cleansing composition.

### BACKGROUND OF THE INVENTION

Combinations of various polymer compounds and oil agents have heretofore been studied for cleansing compositions for use by wiping off makeup or by rinsing off makeup.

For example, Patent Literature 1 discloses a body cleansing composition containing an acrylic acid polymer and an oil agent. Patent Literature 2 discloses a foamable emulsion containing an oil agent, an anionic and/or nonionic polymer, and a cationic surfactant or polymer.
(Patent Literature 1) JP-A-H11-503461
(Patent Literature 2) JP-A-H8-505636

### SUMMARY OF INVENTION

The present invention relates to a skin cleansing composition comprising the following components (A), (B), and (C):

(A) from 0.5 to 40 mass % of an ether oil in a liquid state at 25° C.,
(B) from 0.01 to 5 mass % of a nonionic water-soluble polymer comprising cellulose as a constitutional unit selected from the group consisting of methylcellulose, ethylcellulose, hydroxyethylcellulose, hydroxypropylcellulose, and hydroxypropylmethylcellulose, and
(C) water, and
1 mass % or less of a nonionic surfactant, wherein the mass ratio of the component (B) to the component (A), (B)/(A) is from 0.001 to 5.

### DETAILED DESCRIPTION OF THE INVENTION

The problems of conventional cleansing compositions are that they have an insufficient cleansing power against, for example, traces of stay-put makeup such as oil-based mascara, may further leave a sticky feeling on the skin after wiping or after rinsing without leaving a favorable feel of the skin, and fail to sustain a favorable feel.

The present invention relates to a skin cleansing composition which can thoroughly remove even stay-put makeup such as waterproof mascara and sustains a smooth feel, which is a favorable feel, of the skin after wiping or after rinsing.

The present inventors have found that a skin cleansing composition which solves the problems described above can be obtained by using an ether oil in a liquid state and a nonionic water-soluble polymer in combination at specific ratios.

The skin cleansing composition of the present invention produces a high cleansing power against even stay-put makeup such as waterproof mascara, offers a smooth feel of the skin after cleansing, and sustains fresh, soft, and smooth feels of the skin. In this context, the smooth feel refers to a feel as if the skin is covered with a moderately hydrated film having a moist feeling without stickiness and roughness resulting from dryness.

The skin cleansing composition of the present invention also has excellent dispersibility of a water-soluble polymer and favorable stability.

The ether oil as the component (A) used in the present invention is in a liquid state at 25° C. The liquid state refers to a state having flowability—at 25° C. and represents that the viscosity at 25° C. is 10,000 mPa·s or lower, preferably 50 mPa·s or lower, and more preferably 15 mPa·s or lower.

The ether oil in a liquid state as the component (A) is preferably a dialkyl ether having two alkyl groups each having 16 or less of carbon atom, and more preferably a dialkyl ether having two alkyl groups each having 8 or less of carbon atom. In the dialkyl ether, the two alkyl groups may be the same or different.

More specific examples thereof include dihexyl ether, dioctyl ether, cetyl-1,3-dimethyl butyl ether, and dilauryl ether. Of them, cetyl-1,3-dimethyl butyl ether and dioctyl ether are preferred, and dioctyl ether is more preferred, from the viewpoint of a higher cleansing power and a less oily feeling.

The component (A) can be used alone or in combination of two or more thereof. The content of the component (A) in the total composition is 0.5 mass % or more, preferably 1 mass % or more, and more preferably 1.5 mass % or more, and is 40 mass % or less, preferably 25 mass % or less, and more preferably 10 mass % or less, from the viewpoint of sustaining soft and smooth feelings of the skin after cleansing and also offering a fresh feeling. The content of the component (A) in the total composition is from 0.5 to 40 mass %, preferably from 1 to 25 mass %, and more preferably from 1.5 to 10 mass %.

The mass ratio of the component (A) to the whole oil phase, ((A)/whole oil phase amount)) is preferably 0.05 or more, more preferably 0.08 or more, and further more preferably 0.12 or more, from the viewpoint of the softness of the skin after cleansing. The mass ratio of the component (A) to the whole oil phase, ((A)/whole oil phase amount) is 1 or less.

The nonionic water-soluble polymer as the component (B) is preferably a nonionic water-soluble polymer comprising cellulose as a constitutional unit. Examples of such a water-soluble polymer include: alkylcelluloses such as methylcellulose and ethylcellulose; and hydroxyalkylcelluloses such as hydroxyethylcellulose, hydroxypropylcellulose, and hydroxypropylmethylcellulose.

Of them, a hydroxyalkylcellulose is preferred, and hydroxyethylcellulose is more preferred, from the viewpoint of a high cleansing power and the absence of an oily feeling.

The nonionic water-soluble polymer as the component (B) has a molecular weight of preferably from 50,000 to 3,000,000, and more preferably from 100,000 to 1,600,000, from the viewpoint of a cleansing power, and further more preferably has a molecular weight of from 180,000 to 280,000 from the viewpoint of manufacturability.

The component (B) can be used alone or in combination of two or more thereof. The content of the component (B) in the total composition is 0.01 mass % or more, preferably 0.05 mass % or more, and more preferably 0.075 mass % or more, and is 5 mass % or less, preferably 2 mass % or less, and more preferably 0.5 mass % or less, from the viewpoint of sustaining a soft feeling of the skin after cleansing. The content of the component (B) in the total composition is from 0.01 to 5 mass %, preferably from 0.05 to 2 mass %, and more preferably from 0.075 to 0.5 mass %.

In the present invention, the mass ratio of the component (B) to the component (A), (B)/(A) is 0.001 or more, preferably 0.004 or more, and more preferably 0.01 or more, and is 5 or less, preferably 2.5 or less, and more preferably 1 or less, from the viewpoint of a smooth feel remaining on the skin, the sustention of the smooth feel, and the soft and fresh skin. The mass ratio of the component (B) to the component (A), (B)/(A) is from 0.001 to 5, preferably from 0.004 to 2.5, and more preferably from 0.01 to 1.

The skin cleansing composition of the present invention leaves a smooth feel on the skin after application and also leaves a smooth feel over time, presumably because the combination of the component (A) and the component (B) forms a uniform film on the skin by drying after application to the skin.

For example, when the skin cleansing composition of the present invention is applied onto a glass plate and dried, volatile components vaporize gradually so that mainly the component (A) and the component (B) remain to form a film. In a state where this film is undried, the component (B) is highly dispersible in the component (A) and water, and each component is uniformly dispersed in the composition. As a result, a uniform film is easily formed by drying. This film is made of the ether oil as the component (A) and the nonionic water-soluble polymer as the component (B) in combination and therefore, can further offer a smooth feel when touched with fingers. Hence, the skin cleansing composition of the present invention after application to the skin leaves the film made of the component (A) and the component (B) on the skin. The smooth feel therefore remains on the skin, and the composition is easily adsorbed onto the skin. Therefore, it is regarded that this feature probably allows the smooth feel to be sustained.

In the present invention, the water as the component (C) balances the components. The content of the component (C) in the total composition is preferably 30 mass % or more, more preferably 40 mass % or more, and further more preferably 50 mass % or more, and is preferably 97 mass % or less, more preferably 95 mass % or less, and further more preferably 90 mass % or less.

In the present invention, the mass ratio of the component (A) to the component (C), (A)/(C) is preferably 0.005 or more, more preferably 0.015 or more, and further more preferably 0.02 or more, and is preferably 1 or less, more preferably 0.2 or less, and further more preferably 0.1 or less, from the viewpoint of producing a fresh feel of the skin and the balance between the fresh feel and a feel of the skin after cleansing. The mass ratio of the component (A) to the component (C), (A)/(C) is preferably from 0.005 to 1, more preferably from 0.015 to 0.2, and further more preferably from 0.02 to 0.1.

The cleansing composition of the present invention can further comprise (D) a hydrocarbon oil having a viscosity of 30 mPa·s or lower at 30° C. and, more preferably, can further comprise a hydrocarbon oil having a viscosity of from 1 to 20 mPa·s, further more preferably from 1 to 15 mPa·s, and even more preferably from 1 to 10 mPa·s, at 30° C. The additional use of the component (D) can prevent an oily feeling and improve a fresh cleansed feeling.

In this context, the viscosity is measured using a BM-type viscometer (manufactured by Tokimec, Inc., measurement conditions: rotor No. 1, 60 rpm, 1 min.).

Examples of the hydrocarbon oil as the component (D) include isododecane, isohexadecane, and hydrogenated polyisobutene. Isododecane is preferred from the viewpoint of an excellent cleansing power and an oil-free feeling.

The component (D) can be used alone or in combination of two or more thereof. The content of the component (D) in the total composition is preferably 0.5 mass % or more, more preferably 1 mass % or more, and further more preferably 3 mass % or more, and is preferably 35 mass % or less, more preferably 20 mass % or less, and further more preferably 15 mass % or less, from the viewpoint of a fresh feeling. The content of the component (D) in the total composition is preferably from 0.5 to 35 mass %, more preferably from 1 to 20 mass %, further more preferably from 3 to 15 mass %.

In the present invention, the mass ratio between the component (A) and the component (D), (A)/((A)+(D)) is preferably 0.05 or more, more preferably 0.08 or more, and further more preferably 0.12 or more, and is preferably 0.8 or less, more preferably 0.5 or less, and further more preferably 0.3 or less, from the viewpoint of achieving moist skin after cleansing. The mass ratio between the component (A) and the component (D), (A)/((A)+(D)) is preferably from 0.05 to 0.8, more preferably from 0.08 to 0.5, and further more preferably from 0.12 to 0.3.

The skin cleansing composition of the present invention can further comprise (E) a water-soluble polymer comprising acrylic acid or methacrylic acid as a constitutional unit (water-soluble polymer comprising a constitutional unit derived from acrylic acid or methacrylic acid). The water-soluble polymer can improve stability over time.

The water-soluble polymer comprising acrylic acid or methacrylic acid as a constitutional unit is synthesized from acrylic acid or methacrylic acid as a monomer and is, for example, an acrylic acid/alkyl methacrylate copolymer, preferably a copolymer of acrylic acid and alkyl methacrylate having 10 to 30 of carbon atom ((acrylic acid/alkyl acrylate (C10-30)) copolymer). Examples of commercially available products include, for example, PEMULEN TR-1, PEMULEN TR-2, and CARBOPOL ETD2020 (all manufactured by Lubrizol Advanced Materials, Inc.).

The water-soluble polymer comprising acrylic acid or methacrylic acid as a constitutional unit as the component (E) is preferably used after neutralization of the whole or a portion of the acrylic acid or methacrylic acid units using an alkali agent. The alkali agent for neutralization is not limited as long as the alkali agent can be normally added to cosmetics. For example, potassium hydroxide or sodium hydroxide can be used. The alkali agent can be used alone or in combination of two or more thereof, and is preferably contained at 0.00004 mass % or more and 1.2 mass % or less in the skin cleansing composition of the present invention. It is preferable to adjust the pH of the skin cleansing composition of the present invention to preferably from 5 to 9, and more preferably from 5.5 to 8.

The component (E) can be used alone or in combination of two or more thereof. The content of the component (E) in the total composition is preferably 0.0001 mass % or more, more preferably 0.01 mass % or more, and further more preferably 0.03 mass % or more, and is preferably 3 mass % or less, more preferably 1 mass % or less, and further more preferably 0.3 mass % or less, from the viewpoint of a feeling upon application after preservation and a cleansing power. The content of the component (E) in the total composition is preferably from 0.0001 to 3 mass %, more preferably from 0.01 to 1 mass %, and further more preferably from 0.03 to 0.3 mass %.

The cleansing composition of the present invention can further comprise (F) a water-soluble solvent. The water-soluble solvent can impart a moist feeling.

Specific examples of the component (F) include polyhydric alcohols, polyglycerin, polyethylene glycol, polypropylene glycol, glyceryl ethers having an alkylene group added to glycerin, and sugars.

More specifically, examples of the polyhydric alcohols include ethylene glycol, propylene glycol, isoprene glycol, 1,3-butylene glycol, hexylene glycol, trimethylolpropane, and glycerin. Of them, propylene glycol, isoprene glycol, 1,3-butylene glycol, hexylene glycol, and glycerin are preferred. 1,3-Butylene glycol, propylene glycol, isoprene glycol, and glycerin are more preferred from the viewpoint of a moist feeling of the skin after rinsing or after wiping.

Examples of the polyglycerin, the polyethylene glycol, and the polypropylene glycol include polyglycerin having a molecular weight of 2,000 or smaller, polyethylene glycol having a molecular weight of 10,000 or smaller, and polypropylene glycol having a molecular weight of 1,000 or smaller. Of them, diglycerin, polyethylene glycol 200, polyethylene glycol 400, polyethylene glycol 1000, polyethylene glycol 1540, dipropylene glycol, polypropylene glycol (3), and polypropylene glycol (7) are preferred from the viewpoint of a moist feeling of the skin after rinsing or after wiping.

Examples of the glyceryl ethers having an alkylene group added to glycerin include polyoxypropylene diglyceryl ether, polyoxypropylene glyceryl ether, and polyoxybutylene polyoxyethylene polyoxypropylene glyceryl ether. Of them, polyoxypropylene (9) diglyceryl ether, polyoxypropylene (14) diglyceryl ether, and polyoxybutylene polyoxyethylene polyoxypropylene glyceryl ether (3B.O.)(8E.O.)(5P.O.) are preferred from the viewpoint of low stickiness.

Examples of the sugar include sorbitol, erythritol, pentaerythritol, methyl glucoside, ethyl glucoside, polyoxyethylene methyl glucoside, and polyoxypropylene methyl glucoside. Alkyl glucoside having 2 or less of carbon atom in the alkyl chain is preferred. Of them, methyl glucoside, ethyl glucoside, polyoxyethylene methyl glucoside, and polyoxypropylene methyl glucoside are preferred. Polyoxyethylene methyl glucoside and polyoxypropylene methyl glucoside are more preferred from the viewpoint of a moist feeling of the skin after rinsing or after wiping.

The component (F) can be used alone or in combination of two or more thereof. The content of the component (F) in the total composition is preferably 1 mass % or more, more preferably 3 mass % or more, and further more preferably 4 mass % or more, and is preferably 35 mass % or less, more preferably 25 mass % or less, and further more preferably 15 mass % or less, from the viewpoint of offering a moist feeling and preventing stickiness. The content of the component (F) in the total composition is preferably from 1 to 35 mass %, more preferably from 3 to 25 mass %, and further more preferably from 4 to 15 mass %.

The skin cleansing composition of the present invention can comprise, in addition to the components described above, a component used in ordinary cleansing compositions without inhibiting the effects of the present invention. The skin cleansing composition of the present invention can comprise, for example, an oil agent other than the components (A) and (D), a polymer other than the components (B) and (E), a nonionic surfactant, an anionic surfactant, a cationic surfactant, an amphoteric surfactant, a water-soluble solvent other than the component (F), a bactericide, an anti-inflammatory agent, an antiseptic, a chelating agent, a salt, a pearling agent, a fragrance, a cooling agent, a dye, an ultraviolet absorber, an antioxidant, and a plant extract.

Examples of the oil agent other than the components (A) and (D) include hydrocarbon oils, such as liquid paraffin, having a viscosity exceeding 30 mPa·s at 30° C. The oil agent other than the components (A) and (D) can be used alone or in combination of two or more thereof. The content of the oil agent other than the components (A) and (D) in the total composition is preferably 0.01 mass % or more, and is preferably 20 mass % or less, more preferably 10 mass % or less, further more preferably 5 mass % or less, and even more preferably 1 mass % or less.

Examples of the polymer other than the components (B) and (E) include sodium hyaluronate.

The skin cleansing composition of the present invention can comprise a nonionic surfactant having an HLB of 8 or more, and preferably from 8 to 15. The nonionic surfactant can impart better stability.

In this context, the HLB refers to an index which indicates a hydrophilic-lipophilic balance. In the present invention, the value calculated using the following expression of Oda, Teramura, etc. is used:

$$HLB = \frac{Σ Inorganic value}{Σ Organic value} \times 10$$

Examples of the nonionic surfactant include polyglycerin fatty acid ester, polyethylene glycol fatty acid ester, polyoxyethylene glycerin fatty acid ester, polyoxyethylene polyoxypropylene glycol, polyoxyethylene sorbitan fatty acid ester, polyoxyethylene sorbitol fatty acid ester, polyoxyethylene castor oil, polyoxyethylene hydrogenated castor oil, polyoxyethylene hydrogenated castor oil fatty acid ester, polyoxyethylene alkyl ether, polyoxyethylene alkyl ether fatty acid ester, sucrose fatty acid ester, alkyl polyglucoside, and (poly)alkyl glyceryl ether. Of them, polyglycerin fatty acid ester, polyethylene glycol fatty acid ester, polyoxyethylene glycerin fatty acid ester, polyoxyethylene sorbitan fatty acid ester, and polyoxyethylene hydrogenated castor oil fatty acid ester are preferred from the viewpoint of improving stability.

The nonionic surfactant can be used alone or in combination of two or more thereof. The content of the nonionic surfactant in the total composition is preferably 0.01 mass % or more, and is preferably 5 mass % or less, more preferably 1 mass % or less, and further more preferably 0.5 mass % or less.

The ratio of the nonionic surfactant to the total amount of oil agents, (nonionic surfactant amount/the total amount of oil agents), is preferably 0.1 or less, more preferably 0.01 or less, and further more preferably 0.005 or less.

The skin cleansing composition of the present invention is preferably free from the anionic surfactant, the cationic surfactant, and the amphoteric surfactant, except when these surfactants are irreversibly mixed thereinto insofar as not inhibiting the effects of the present invention.

The skin cleansing composition of the present invention can be produced according to a conventional method by mixing the components to be contained. The skin cleansing composition comprising a solid material at normal temperature can be produced by thermally melting the solid material or dissolving the solid material in other components and then uniformly mixing all components.

The skin cleansing composition of the present invention has a viscosity of preferably from 200 to 10,000 mPa·s, and more preferably from 500 to 4,000 mPa·s, at 25° C. from the viewpoint of low stickiness and a feeling upon application after preservation.

The skin cleansing composition of the present invention is suitable as, for example, a facial wash or a cleansing agent and is more preferably a cleansing agent. Also, the skin cleansing composition of the present invention is preferably used for cleansing a makeup cosmetic applied on the face.

The cleansing composition of the present invention can be used by, for example, a method of applying the composition on a sheet etc. and wiping the skin with the sheet etc., where the composition may be used as it is or after shaking it up till uniform if it is a separated composition;

a method of wiping with a sheet etc. after applying the composition to the skin with a palm;
a method of washing off after applying the composition to the skin with a palm;
a method of washing off after applying the composition to a sheet etc. and spreading the composition over the skin;
a method of washing off the composition after wiping the skin according to any one of the above methods. The composition can be also used in a form in which a sheet is impregnated with the composition.

In relation to the embodiments mentioned above, the present invention further discloses the following compositions.

<1> A skin cleansing composition comprising the following components (A), (B), and (C):
(A) from 0.5 to 40 mass % of an ether oil in a liquid state at 25° C.,
(B) from 0.01 to 5 mass % of a nonionic water-soluble polymer comprising cellulose as a constitutional unit selected from the group consisting of methylcellulose, ethylcellulose, hydroxyethylcellulose, hydroxypropylcellulose, and hydroxypropylmethylcellulose, and
(C) water, and
1 mass % or less of a nonionic surfactant, wherein the mass ratio of the component (B) to the component (A), (B)/(A) is from 0.001 to 5.
<2> The skin cleansing composition according to <1>, wherein the component (A) is preferably a dialkyl ether having two alkyl groups each having 16 or less of carbon atom, and more preferably a dialkyl ether having two alkyl groups each having 8 or less of carbon atom.
<3> The skin cleansing composition according to <1> or <2>, wherein the component (A) is preferably dihexyl ether, dioctyl ether, cetyl-1,3-dimethyl butyl ether, or dilauryl ether, more preferably cetyl-1,3-dimethyl butyl ether or dioctyl ether, and further more preferably dioctyl ether.
<4> The skin cleansing composition according to any one of <1> to <3>, wherein the content of the component (A) in the total composition is preferably 1 mass % or more, more preferably 1.5 mass % or more, and is preferably 25 mass % or less, more preferably 10 mass % or less, and the content of the component (A) in the total composition is preferably from 1 to 25 mass %, and more preferably from 1.5 to 10 mass %.
<5> The skin cleansing composition according to any one of <1> to <4>, wherein the nonionic water-soluble polymer as the component (B) is preferably a nonionic water-soluble polymer comprising cellulose as a constitutional unit, more preferably methylcellulose, ethylcellulose, hydroxyethylcellulose, hydroxypropylcellulose, or hydroxypropylmethylcellulose, and further more preferably hydroxyethylcellulose.
<6> The skin cleansing composition according to any one of <1> to <5>, wherein the content of the component (B) in the total composition is preferably 0.05 mass % or more, more preferably 0.075 mass % or more, and is preferably 2 mass % or less, more preferably 0.5 mass % or less, and the content of the component (B) in the total composition is preferably from 0.05 to 2 mass %, and more preferably from 0.075 to 0.5 mass %.
<7> The skin cleansing composition according to any one of <1> to <6>, wherein the mass ratio of the component (A) to the whole oil phase, ((A)/whole oil phase amount)), is preferably 0.05 or more, more preferably 0.08 or more, and further more preferably 0.12 or more.<8> The skin cleansing composition according to any one of <1> to <7>, wherein the mass ratio of the component (B) to the component (A), (B)/(A) is preferably 0.004 or more, more preferably 0.01 or more, and is preferably 2.5 or less, more preferably 1 or less, and the mass ratio of the component (B) to the component (A), (B)/(A) is preferably from 0.004 to 2.5, and more preferably from 0.01 to 1.
<9> The skin cleansing composition according to any one of <1> to <8>, wherein the content of the component (C) in the total composition is preferably 30 mass % or more, more preferably 40 mass % or more, and further more preferably 50 mass % or more, and is preferably 97 mass % or less, more preferably 95 mass % or less, and further more preferably 90 mass % or less.
<10> The skin cleansing composition according to any one of <1> to <9>, further comprising (D) a hydrocarbon oil having a viscosity of 30 mPa·s or lower, preferably from 1 to 20 mPa·s, more preferably from 1 to 15 mPa·s, and further more preferably from 1 to 10 mPa·s, at 30° C.
<11> The skin cleansing composition according to <10>, wherein the content of the component (D) in the total composition is preferably 0.5 mass % or more, more preferably 1 mass % or more, and further more preferably 3 mass % or more, and is preferably 35 mass % or less, more preferably 20 mass % or less, and further more preferably 15 mass % or less, and the content of the component (D) in the total composition is preferably from 0.5 to 35 mass %, more preferably from 1 to 20 mass %, and further more preferably from 3 to 15 mass %.
<12> The skin cleansing composition according to <10> or <11>, wherein the component (D) is preferably isododecane, isohexadecane, or hydrogenated polyisobutene, and more preferably isododecane.
<13> The skin cleansing composition according to any one of <1> to <12>, further comprising (E) a water-soluble polymer comprising acrylic acid or methacrylic acid as a constitutional unit (water-soluble polymer comprising a constitutional unit derived from acrylic acid or methacrylic acid).
<14> The skin cleansing composition according to <13>, wherein the component (E) is synthesized from acrylic acid or methacrylic acid as a monomer and is preferably an acrylic acid/alkyl methacrylate copolymer, more preferably a copolymer of acrylic acid and alkyl methacrylate having 10 to 30 of carbon atom.
<15> The skin cleansing composition according to <13> or <14>, wherein the content of the component (E) in the total composition is preferably 0.0001 mass % or more, more preferably 0.01 mass % or more, and further more preferably 0.03 mass % or more, and is preferably 3 mass % or less, more preferably 1 mass % or less, and further more preferably 0.3 mass % or less. The content of the component (E) in the total composition is preferably from 0.0001 to 3 mass %, more preferably from 0.01 to 1 mass %, and further more preferably 0.03 to 0.3 mass %.
<16> The skin cleansing composition according to any one of <1> to <15>, further comprising (F) a water-soluble solvent.
<17> The skin cleansing composition according to <16>, wherein (F) the water-soluble solvent is preferably propylene glycol, isoprene glycol, 1,3-butylene glycol, hexylene glycol, or glycerin, and more preferably 1,3-butylene glycol, propylene glycol, isoprene glycol, glycerin, diglycerin, polyethylene glycol 200, polyethylene glycol 400, polyethylene glycol 1000, polyethylene glycol 1540, dipropylene glycol, polypropylene glycol (3), polypropylene glycol (7), polyoxypropylene (9) diglyceryl ether, polyoxypropylene (14) diglyceryl ether, polyoxybutlene polyoxyethylene polyoxypropylene glyceryl ether (3B.O.)(8E.O.)(5P.O.), polyoxyethylene methyl glucoside, or polyoxypropylene methyl glucoside.
<18> The skin cleansing composition according to <16> or <17>, wherein the content of the component (F) in the total composition is preferably 1 mass % or more, more preferably 3 mass % or more, and further more preferably 4 mass % or more, and is preferably 35 mass % or less, more preferably 25 mass % or less, and further more preferably 15 mass % or less, and the content′ of the component (F) in the total composition is preferably from 1 to 35 mass %, more preferably from 3 to 25 mass %, and further more preferably from 4 to 15 mass %.
<19> The skin cleansing composition according to any one of <1> to <18>, wherein the mass ratio of the component (A) to the component (C), (A)/(C) is preferably 0.005 or more, more preferably 0.015 or more, and further more preferably 0.02 or more, and is preferably 1 or less, more preferably 0.2 or less, and further more preferably 0.1 or less, and the mass ratio of the component (A) to the component (C), (A)/(C) is preferably from 0.005 to 1, more preferably from 0.015 to 0.2, and further more preferably from 0.02 to 0.1.
<20> The skin cleansing composition according to any one of <10> to <19>, wherein the mass ratio between the component (A) and the component (D), (A)/((A)+(D)) is preferably 0.05 or more, more preferably 0.08 or more, and further more preferably 0.12 or more, and is preferably 0.8 or less, more preferably 0.5 or less, and further more preferably 0.3 or less, and the mass ratio between the component (A) and the component (D), (A)/((A)+(D)) is preferably from 0.05 to 0.8, more preferably from 0.08 to 0.5, and further more preferably from 0.12 to 0.3.
<21> The skin cleansing composition according to any one of <1> to <20>, wherein the content of an oil agent other than the components (A) and (D) in the total composition is preferably 0.01 mass % or more, and is preferably 20 mass % or less, more preferably 10 mass % or less, further more preferably 5 mass % or less, and even more preferably 1 mass % or less.
<22> The skin cleansing composition according to any one of <1> to <21>, wherein the nonionic surfactant has an HLB of preferably 8 or more, and more preferably from 8 to 15.
<23> The skin cleansing composition according to any one of <1> to <22>, wherein the nonionic surfactant is preferably polyglycerin fatty acid ester, polyethylene glycol fatty acid ester, polyoxyethylene glycerin fatty acid ester, polyoxyethylene sorbitan fatty acid ester, or polyoxyethylene hydrogenated castor oil fatty acid ester.
<24> The skin cleansing composition according to any one of <1> to <23>, wherein the content of the nonionic surfactant in the total composition is preferably 0.01 mass % or more, and is preferably 0.5 mass % or less.
<25> The skin cleansing composition according to any one of <1> to <24>, wherein the ratio of the nonionic surfactant to the total amount of oil agents, (nonionic surfactant amount/total amount of oil agents), is preferably 0.1 or less, more preferably 0.01 or less, and further more preferably 0.005 or less.
<26> The skin cleansing composition according to any one of <1> to <25>, wherein the skin cleansing composition preferably contains none of an anionic surfactant, a cationic surfactant and an amphoteric surfactant.
<27> The skin cleansing composition according to any one of <1> to <26>, wherein the skin cleansing composition is suitable for one selected from the group consisting of a facial wash and a cleansing agent, more preferably a cleansing agent, and is preferably used for cleansing a makeup cosmetic applied on the face.
<28> The cleansing composition according to any one of <1> to <27>, wherein the cleansing composition can be used by a method of applying the composition on a sheet and wiping the skin with the sheet, where the composition may be used as it is or after shaking it up till uniform if it is a separated composition; a method of wiping with a sheet after applying the composition to the skin with a palm; a method of washing off after applying the composition to the skin with a palm; a method of washing after applying the composition to a sheet and spreading the composition over the skin; or a method of washing off the composition after wiping the skin according to any one of the above methods.
<29> The skin cleansing composition according to any one of <1> to <28>, wherein the method of using the skin cleansing composition in the form of a sheet impregnated therewith is more preferred.
<30> The skin cleansing composition according to <28> or <29>, wherein the sheet preferably comprises cotton.
<31> Use of a skin cleansing composition according to any one of <1> to <30> for the production of a cleansing agent for cleansing a makeup cosmetic applied on the face.
<32> Use of a skin cleansing composition according to any one of <1> to <30> for the production of a cleansing agent for cleansing a makeup cosmetic applied on the face, wherein the cleansing agent is used such that a sheet is impregnated with the cleansing agent and the face is wiped with the sheet.
<33> Use of a skin cleansing composition according to any one of <1> to <30> for the production of a cleansing agent for cleansing a makeup cosmetic applied on the face, wherein the cleansing agent is used in the form of a sheet impregnated therewith.
<34> A skin cleansing composition comprising the following components (A), (B), (C), and (D):
(A) from 0.5 to 40 mass % of an ether oil in a liquid state at 25° C.,
(B) from 0.01 to 5 mass % of a nonionic water-soluble polymer comprising cellulose as a constitutional unit selected from the group consisting of methylcellulose, ethylcellulose, hydroxyethylcellulose, hydroxypropylcellulose, and hydroxypropylmethylcellulose,
(C) water, and
(D) a hydrocarbon oil having a viscosity of 30 mPa·s or lower at 30° C., wherein the mass ratio of the component (B) to the component (A), (B)/(A) is from 0.001 to 5, and
the mass ratio between the component (A) and the component (D), (A)/((A)+(D)) is 0.05 to 0.8.

### EXAMPLES

### Examples 1 to 3 and Comparative Examples 1 and 2

Each skin cleansing composition was produced according to the composition shown in Table 1 and evaluated for the dispersibility of oil, the shape of a film, the sustention of a smooth feel of the skin after wiping, the sustention of the softness of the skin after wiping, and the cleansing power against oil-based mascara. The results are shown in Table 1.

### (Production Method)

The component (A) was added to water such that the total amount of all components might be 100 g. The mixture was stirred at 1,200 rpm for 10 minutes using Disper. Then, the oil agents were added thereto, and the mixture was stirred at 200 rpm for 5 minutes using a propeller stirrer to produce a skin cleansing composition. The skin cleansing composition was produced such that the viscosity might be from 1,000 to 2,500 mPa·s.

### (Evaluation Method)

(1) Dispersibility of oil: (three-component system of oil agent, nonionic polymer having cellulose main chain, and water in Examples 1 to 3 and Comparative Examples 1 and 2)

Each obtained skin cleansing composition was transferred to a 100-mL glass bottle (diameter: approximately 5 cm, height: approximately 8 cm, manufacturer: TGK Co., Ltd.). The glass bottle was shaken up and down by hand for 10 seconds at a rate of 3 reciprocal motions per second and left standing at room temperature. The time required for the oil agents to separate by 1 mm from homogeneous appearance was measured, followed by evaluation according to the following criteria:

5: Longer than 20 seconds.
4: 15 to 20 seconds.
3: 10 to 14 seconds.
2: 5 to 9 seconds.
1: Shorter than 5 seconds.

### (2) Shape of Film:

0.1 g of each obtained skin cleansing composition was added dropwise onto a glass plate within 5 seconds immediately after the production and left standing at room temperature for 1 day for drying. Five expert panelists touched the obtained film-like substance with their fingers and conducted sensory evaluation for its touch to the skin (smooth feel) according to five levels given below. The sum of the scores of the five panelists was used for determination.

### 5: Smooth.

4: Slightly smooth.

### 3: Neutral.

2: Not so smooth.
1: Not smooth at all.
(3) Sustention of Smooth Feel of Skin after Wiping:

Five expert panelists applied 4 g of each cleansing composition uniformly on 1 g of cotton nonwoven and then wiped their foundation (Sofina Primavista Liquid Foundation Ocher 05; the same foundation was used in subsequent evaluations)—applied faces using the nonwoven. The panelists kept quiet for 3 hours in a room of 30° C. and 60% RH and then conducted sensory evaluation for a smooth feel of the skin according to five levels given below. The sum of the scores of the five panelists was used for determination.

5: Smooth feel was sustained.
4: Slightly smooth feel was sustained.

### 3: Neutral.

2: Smooth feel was hardly sustained.
1: No smooth feel was sustained.
(4) Sustention of Softness of Skin after Wiping:

Five expert panelists applied 4 g of each cleansing composition uniformly on 1 g of cotton nonwoven and then wiped their foundation-applied faces using the nonwoven. The panelists kept quiet for 3 hours in a room of 30° C. and 60% RH and then conducted sensory evaluation for the softness of the skin according to five levels given below. The sum of the scores of the five panelists was used for determination.

### 5: Soft.

4: Slightly soft.

### 3: Neutral.

2: Not so soft.
1: Not soft.

### (5) Cleansing Power Against Oil-Based Mascara:

Five expert panelists uniformly applied 0.005 g of KOSE Sports Beauty Fasio Power Stay Mascara (Curl Long) BK001 (trade name) as oil-based mascara (waterproof mascara) in a circular pattern of 1.2 cm in diameter onto slide glass, which was then left for 2 hours for drying. The oil-based mascara was lightly rubbed for 10 seconds by finger massage using approximately 0.05 g of each cleansing composition and then wiped off with cotton at a fixed pressure (7.8 kPa). The panelists evaluated the removal of the mascara according to five levels given below. The sum of the scores of the five panelists was used for determination.

5: Removed very well.
4: Removed well.

### 3: Neutral.

2: Not removed very well.
1: Not removed well.

TABLE 1

| | | | | | Comparative | |
|---|---|---|---|---|---|---|
| | | Example | | | Example | |
| Component (mass %) | | 1 | 2 | 3 | 1 | 2 |
| A | Dioctyl ether*¹ | 15 | 15 | 15 | | |
| | Isododecane*² (3 mPa · s) | | | | 15 | |
| | Isopropyl myristate*³ | | | | | 15 |
| B | Hydroxyethylcellulose*⁴ | 5 | 2 | 0.5 | 5 | 5 |
| C | Water | 80 | 83 | 84.5 | 80 | 80 |

| | | | | | |
|---|---|---|---|---|---|
| Total | 100 | 100 | 100 | 100 | 100 |
| B/A | 0.33 | 0.13 | 0.03 | — | — |
| A/C | 0.19 | 0.18 | 0.18 | 0.00 | 0.00 |
| Dispersibility of oil | 5 | 5 | 5 | 2 | 5 |
| Shape of film | 20 | 23 | 23 | 9 | 19 |
| Sustention of smooth feel of skin after wiping | 19 | 19 | 23 | 9 | 18 |
| Sustention of softness of skin after wiping | 21 | 21 | 24 | 8 | 20 |
| Cleansing power against oil-based mascara | 23 | 23 | 25 | 19 | 10 |

*¹dioctyl ether: CETIOL OE (manufactured by BASF Japan Ltd.)
*²isododecane: MARUKASOL R (manufactured by Maruzen Petrochemical Co., Ltd.)
*³isopropyl myristate (manufactured by Kao Corp.)
*⁴hydroxyethylcellulose 1: HEC Daicel SE400 (manufactured by Daicel FineChem Ltd., molecular weight (Mw): 250,000)

The skin cleansing compositions of Examples 1 to 3 were excellent in the dispersibility of oil and the shape of a film. These compositions were also excellent in the sustention of a smooth feel of the skin after wiping, the sustention of the softness of the skin after wiping, and the cleansing power against oil-based mascara. In contrast, the skin cleansing composition of Comparative Example 1 containing isododecane instead of the component (A) was inferior in the dispersibility of oil and also inferior in the feel of the skin (smooth feel) attributed to the shape of the dried film, the sustention of a smooth feel of the skin after wiping, and the sustention of the softness of the skin after wiping. The skin cleansing composition of Comparative Example 2 containing isopropyl myristate instead of the component (A) was inferior in the cleansing power against oil-based mascara, though having the excellent dispersibility in the film.

### Examples 4 to 16 and Comparative Examples 3 to 10

Each skin cleansing composition was produced according to the composition shown in Tables 2 and 3. The skin cleansing composition was evaluated for the sustention of a smooth feel of the skin after wiping, the sustention of the softness of the skin after wiping, and the cleansing power against oil-based mascara in the same way as in Examples 1 to 3 and also evaluated for the sleekness of the skin after wiping, the fresh feeling after wiping, and the moist feeling after wiping. The results are shown in Tables 2 and 3.

### (Production Method)

Water was placed in a mixing vessel at room temperature. The component (B) was added thereto with stirring using Disper, and the mixture was stirred. If necessary, the component (E) was added thereto simultaneously with the component (B), and the mixture was stirred. Then, a base was added thereto, and the mixture was stirred. The component (A) and other components were further added thereto, and the mixture was stirred using a homomixer to produce a skin cleansing composition. In the case of adding a solid component at normal temperature, the component was thermally melted and then added to the mixing vessel, and the skin cleansing composition was produced in the same way as above, if necessary with heating, and then cooled to room temperature. In this production, the total amount of all components was 1 kg.

### (Evaluation Method)

(1) Sleekness of Skin after Wiping:

Five expert panelists applied 4 g of each cleansing composition uniformly on 1 g of cotton nonwoven and then wiped their foundation-applied faces using the nonwoven, followed by drying for 15 minutes. Then, the panelists conducted sensory evaluation for the sleekness of the skin according to five levels given below. The sum of the scores of the five panelists was used for determination.

### 5: Sleek.

4: Slightly sleek.

### 3: Neutral.

2: Not very sleek.
1: Not sleek.
(2) Fresh Feeling after Wiping:

Five expert panelists applied 4 g of each cleansing composition uniformly on 1 g of cotton nonwoven fabric and then wiped their foundation-applied faces using the nonwoven. Then, the panelists conducted sensory evaluation for the fresh feeling according to five levels given below. The sum of the scores of the five panelists was used for determination.

### 5: Fresh.

4: Slightly fresh.

### 3: Neutral.

2: Not very fresh.
1: Not fresh.
(3) Moist Feeling after Wiping:

Five expert panelists applied 4 g of each cleansing composition uniformly on 1 g of cotton nonwoven and then wiped their foundation-applied faces using the nonwoven. Then, the panelists conducted sensory evaluation for the moist feeling according to five levels given below. The sum of the scores of the five panelists was used for determination.

### 5: Moist.

4: Slightly moist.

### 3: Neutral.

2: Not very moist.
1: Not moist.

TABLE 2

| | | Example | | | | | | | | | | |
|---|---|---|---|---|---|---|---|---|---|---|---|---|
| Component (mass %) | | 4 | 5 | 6 | 7 | 8 | 9 | 10 | 11 | 12 | 13 | 14 |
| A | Dioctyl ether*¹ | 0 | 14 | 14 | 2 | 2 | 2 | 2 | 1 | 28 | 28 | 38 |
| | Cetyl-1,3-dimethyl butyl ether*⁵ | 14 | 0 | 0 | 0 | 0 | 0 | 0 | 0 | 0 | 0 | 0 |
| B | Hydroxyethylcellulose 1*⁴ | 0.3 | 0.3 | 0 | 0.3 | 0 | 2 | 5 | 5 | 0.1 | 0.05 | 0.05 |
| | Hydroxyethylcellulose 2*⁶ | 0 | 0 | 0 | 0 | 0.1 | 0 | 0 | 0 | 0 | 0 | 0 |
| | Hydroxypropylmethylcellulose*⁷ | 0 | 0 | 0.3 | 0 | 0 | 0 | 0 | 0 | 0 | 0 | 0 |
| C | Water | 85.148 | 85.148 | 85.148 | 85.148 | 75.36 | 74.448 | 71.448 | 72.448 | 50.348 | 50.398 | 40.398 |
| D | Isododecane*² (3 mPa · s) | 0 | 0 | 0 | 10 | 12 | 12 | 12 | 12 | 12 | 12 | 12 |
| | Isohexadecane*⁸ (5 mPa · s) | 0 | 0 | 0 | 1 | 0 | 0 | 0 | 0 | 0 | 0 | 0 |
| | Hydrogenated polyisobutene*⁹ | 0 | 0 | 0 | 1 | 0 | 0 | 0 | 0 | 0 | 0 | 0 |
| | (14 mPa · s) | | | | | | | | | | | |
| E | Acrylic acid/alkyl methacrylate | 0.075 | 0.075 | 0.075 | 0.075 | 0.075 | 0.075 | 0.075 | 0.075 | 0.075 | 0.075 | 0.075 |
| | copolymer*¹⁰ | | | | | | | | | | | |
| | Sodium hydroxyethylcellulose | 0 | 0 | 0 | 0 | 0 | 0 | 0 | 0 | 0 | 0 | 0 |
| | hydroxypropyl stearyl ether | | | | | | | | | | | |
| | hydroxypropylsulfonate*¹¹ | | | | | | | | | | | |
| F | Polyoxypropylene (9) diglyceryl | 0 | 0 | 0 | 0 | 4 | 4 | 4 | 4 | 4 | 4 | 4 |
| | ether*¹² | | | | | | | | | | | |
| | Polyoxybutylene polyoxyethylene | 0 | 0 | 0 | 0 | 1 | 0 | 0 | 0 | 0 | 0 | 0 |
| | polyoxypropylene glyceryl ether | | | | | | | | | | | |
| | (3B.O.)(8E.O.)(5P.O.)*¹³ | | | | | | | | | | | |
| | 1,3-Butylene glycol | 0 | 0 | 0 | 0 | 5 | 5 | 5 | 5 | 5 | 5 | 5 |
| Others | Polyethylene glycol (12) | 0.045 | 0.045 | 0.045 | 0.045 | 0.045 | 0.045 | 0.045 | 0.045 | 0.045 | 0.045 | 0.045 |
| | monolaurate*¹⁴ | | | | | | | | | | | |
| | (HLB = 14) | | | | | | | | | | | |
| | Potassium hydroxide solution | 0.032 | 0.032 | 0.032 | 0.032 | 0.02 | 0.032 | 0.032 | 0.032 | 0.032 | 0.032 | 0.032 |
| | (48%) | | | | | | | | | | | |
| | Methyl p-hydroxybenzoate | 0.1 | 0.1 | 0.1 | 0.1 | 0.1 | 0.1 | 0.1 | 0.1 | 0.1 | 0.1 | 0.1 |
| | Phenoxyethanol | 0.3 | 0.3 | 0.3 | 0.3 | 0.3 | 0.3 | 0.3 | 0.3 | 0.3 | 0.3 | 0.3 |
| Total | | 100 | 100 | 100 | 100 | 100 | 100 | 100 | 100 | 100 | 100 | 100 |
| B/A | | 0.02 | 0.02 | 0.02 | 0.15 | 0.05 | 1.00 | 2.50 | 5.00 | 0.004 | 0.002 | 0.001 |
| Ratio of A to whole oil phase | | 1.00 | 1.00 | 1.00 | 0.14 | 0.14 | 0.14 | 0.14 | 0.08 | 0.700 | 0.700 | 0.760 |
| A/C | | 0.16 | 0.16 | 0.16 | 0.02 | 0.03 | 0.03 | 0.03 | 0.01 | 0.56 | 0.56 | 0.94 |
| A + D | | 14.0 | 14.0 | 14.0 | 14.0 | 14.0 | 14.0 | 14.0 | 13.0 | 40.0 | 40.0 | 50.0 |
| A/((A) + (D)) | | 1.00 | 1.00 | 1.00 | 0.14 | 0.14 | 0.14 | 0.14 | 0.08 | 0.70 | 0.70 | 0.76 |
| Evalua- | Sustention of smooth feel of | 22 | 23 | 20 | 22 | 23 | 23 | 23 | 23 | 21 | 20 | 20 |
| tion | skin after wiping | | | | | | | | | | | |
| | Sustention of softness of skin | 20 | 23 | 21 | 23 | 23 | 21 | 20 | 19 | 21 | 21 | 21 |
| | after wiping | | | | | | | | | | | |
| | Sleekness of skin after wiping | 24 | 24 | 24 | 24 | 24 | 23 | 22 | 23 | 24 | 24 | 24 |
| | Fresh feeling after wiping | 18 | 20 | 20 | 24 | 25 | 25 | 25 | 21 | 20 | 18 | 16 |
| | Moist feeling after wiping | 20 | 20 | 20 | 19 | 24 | 24 | 24 | 20 | 25 | 25 | 25 |
| | Cleansing power against | 20 | 23 | 21 | 23 | 20 | 20 | 20 | 20 | 24 | 24 | 24 |
| | oil-based mascara | | | | | | | | | | | |

*¹dioctyl ether: CETIOL OE (manufactured by BASF Japan Ltd.)
*²isododecane: MARUKASOL R (manufactured by Maruzen Petrochemical Co., Ltd.)
*⁴hydroxyethylcellulose 1: HEC Daicel SE400 (manufactured by Daicel FineChem Ltd., molecular weight (Mw): 250,000)
*⁵cetyl-1,3-dimethyl butyl ether: ASE-166K (manufactured by Kao Corp.)
*⁶hydroxyethylcellulose 2: HEC Daicel SE900 (manufactured by Daicel FineChem Ltd., molecular weight (Mw): 1,560,000)
*⁷hydroxypropylmethylcellulose: METOLOSE 60SH-4000 (manufactured by Shin-Etsu Chemical Co., Ltd., molecular weight (Mw): 200,000)
*⁸isohexadecane: PARLEAM 4 (manufactured by NOF Corp.)
*⁹hydrogenated polyisobutene: PARLEAM EX (manufactured by NOF Corp.)
*¹⁰acrylic acid/alkyl methacrylate copolymer: PEMULEN TR-2 (manufactured by Lubrizol Advanced Materials, Inc.)
*¹¹sodium hydroxyethylcellulose hydroxypropyl stearyl ether hydroxypropylsulfonate: SPS-S-SA (manufactured by Kao Corp.)
*¹²polyoxypropylene (9) diglyceryl ether: SY-DP9 (manufactured by Sakamoto Yakuhin Kogyo Co., Ltd.)
*¹³polyoxybutylene polyoxyethylene polyoxypropylene glyceryl ether (3B.O.)(8E.O.)(5P.O.): WILBRIDE S-753 (manufactured by NOF Corp.)
*¹⁴polyethylene glycol (12) monolaurate: EMANON 1112 (manufactured by Kao Corp.)

TABLE 3

| | | Example | | Comparative Example | | | | | | | |
|---|---|---|---|---|---|---|---|---|---|---|---|
| Component (mass %) | | 15 | 16 | 3 | 4 | 5 | 6 | 7 | 8 | 9 | 10 |
| A | Dioctyl ether*¹ | 3 | 2 | 0 | 2 | 0 | 14 | 0 | 13 | 0.1 | 0.5 |
| | Cetyl-1,3-dimethyl butyl ether*⁵ | 0 | 0 | 0 | 0 | 0 | 0 | 0 | 0 | 0 | 0 |
| B | Hydroxyethylcellulose 1*⁴ | 0.1 | 0.1 | 0 | 0 | 0.1 | 0 | 0.1 | 0.01 | 2.5 | 5 |
| | Hydroxyethylcellulose 2*⁶ | 0 | 0 | 0 | 0 | 0 | 0 | 0 | 0 | 0 | 0 |
| | Hydroxypropylmethylcellulose*⁷ | 0 | 0 | 0 | 0 | 0 | 0 | 0 | 0 | 0 | 0 |
| C | Water | 75.348 | 76.348 | 76.395 | 76.448 | 76.348 | 76.448 | 90.348 | 76.438 | 84.948 | 71.448 |
| D | Isododecane*² (3 mPa · s) | 12 | 12 | 14 | 12 | 14 | 0 | 0 | 1 | 2.9 | 13.5 |
| | Isohexadecane*⁸ (5 mPa · s) | 0 | 0 | 0 | 0 | 0 | 0 | 0 | 0 | 0 | 0 |
| | Hydrogenated polyisobutene*⁹ | 0 | 0 | 0 | 0 | 0 | 0 | 0 | 0 | 0 | 0 |
| | (14 mPa · s) | | | | | | | | | | |
| E | Acrylic acid/alkyl methacrylate | 0.075 | 0.075 | 0.047 | 0.075 | 0.075 | 0.075 | 0.075 | 0.075 | 0.075 | 0.075 |
| | copolymer*¹⁰ | | | | | | | | | | |
| | Sodium hydroxyethylcellulose | 0 | 0 | 0.093 | 0 | 0 | 0 | 0 | 0 | 0 | 0 |
| | hydroxypropyl stearyl ether | | | | | | | | | | |
| | hydroxypropylsulfonate*¹¹ | | | | | | | | | | |
| F | Polyoxypropylene (9) diglyceryl | 4 | 4 | 4 | 4 | 4 | 4 | 4 | 4 | 4 | 4 |
| | ether*¹² | | | | | | | | | | |
| | Polyoxybutylene polyoxyethylene | 0 | 0 | 0 | 0 | 0 | 0 | 0 | 0 | 0 | 0 |
| | polyoxypropylene glyceryl ether | | | | | | | | | | |
| | (3B.O.)(8E.O.)(5P.O.)*¹³ | | | | | | | | | | |
| | 1,3-Butylene glycol | 5 | 5 | 5 | 5 | 5 | 5 | 5 | 5 | 5 | 5 |
| Others | Polyethylene glycol (12) | 0.045 | 0.045 | 0.045 | 0.045 | 0.045 | 0.045 | 0.045 | 0.045 | 0.045 | 0.045 |
| | monolaurate*¹⁴ | | | | | | | | | | |
| | (HLB = 14) | | | | | | | | | | |
| | Potassium hydroxide solution | 0.032 | 0.032 | 0.02 | 0.032 | 0.032 | 0.032 | 0.032 | 0.032 | 0.032 | 0.032 |
| | (48%) | | | | | | | | | | |
| | Methyl p-hydroxybenzoate | 0.1 | 0.1 | 0.1 | 0.1 | 0.1 | 0.1 | 0.1 | 0.1 | 0.1 | 0.1 |
| | Phenoxyethanol | 0.3 | 0.3 | 0.3 | 0.3 | 0.3 | 0.3 | 0.3 | 0.3 | 0.3 | 0.3 |
| Total | | 100 | 100 | 100 | 100 | 100 | 100 | 100 | 100 | 100 | 100 |
| B/A | | 0.03 | 0.05 | — | 0.00 | — | 0.00 | — | 0.0008 | 25.00 | 10.00 |
| Ratio of A to whole oil phase | | 0.20 | 0.14 | 0.000 | 0.14 | 0.000 | 1.00 | — | 0.9286 | 0.03 | 0.04 |
| A/C | | 0.04 | 0.03 | 0.00 | 0.03 | 0.00 | 0.18 | 0.00 | 0.17 | 0.001 | 0.01 |
| A + D | | 15.0 | 14.0 | 14.0 | 14.0 | 14.0 | 14.0 | 0.0 | 14.0 | 3.0 | 14.0 |
| A/((A) + (D)) | | 0.20 | 0.14 | 0.00 | 0.14 | 0.00 | 1.00 | — | 0.93 | 0.03 | 0.04 |
| Evalua- | Sustention of smooth feel of | 21 | 25 | 9 | 10 | 9 | 11 | 8 | 13 | 14 | 13 |
| tion | skin after wiping | | | | | | | | | | |
| | Sustention of softness of skin | 21 | 25 | 8 | 9 | 8 | 10 | 8 | 12 | 11 | 11 |
| | after wiping | | | | | | | | | | |
| | Sleekness of skin after wiping | 24 | 24 | 11 | 19 | 11 | 19 | 11 | 22 | 12 | 12 |
| | Fresh feeling after wiping | 25 | 25 | 23 | 20 | 23 | 8 | 21 | 15 | 12 | 12 |
| | Moist feeling after wiping | 25 | 25 | 17 | 22 | 17 | 22 | 15 | 21 | 19 | 13 |
| | Cleansing power against | 25 | 25 | 18 | 24 | 18 | 24 | 5 | 22 | 12 | 18 |
| | oil-based mascara | | | | | | | | | | |

*¹dioctyl ether: CETIOL OE (manufactured by BASF Japan Ltd.)
*²isododecane: MARUKASOL R (manufactured by Maruzen Petrochemical Co., Ltd.)
*⁴hydroxyethylcellulose 1: HEC Daicel SE400 (manufactured by Daicel FineChem Ltd., molecular weight (Mw): 250,000)
*⁵cetyl-1,3-dimethyl butyl ether: ASE-166K (manufactured by Kao Corp.)
*⁶hydroxyethylcellulose 2: HEC Daicel SE900 (manufactured by Daicel FineChem Ltd., molecular weight (Mw): 1,560,000)
*⁷hydroxypropylmethylcellulose: METOLOSE 60SH-4000 (manufactured by Shin-Etsu Chemical Co., Ltd., molecular weight (Mw): 200,000)
*⁸isohexadecane: PARLEAM 4 (manufactured by NOF Corp.)
*⁹hydrogenated polyisobutene: PARLEAM EX (manufactured by NOF Corp.)
*¹⁰acrylic acid/alkyl methacrylate copolymer: PEMULEN TR-2 (manufactured by Lubrizol Advanced Materials, Inc.)
*¹¹sodium hydroxyethylcellulose hydroxypropyl stearyl ether hydroxypropylsulfonate: SPS-S-SA (manufactured by Kao Corp.)
*¹²polyoxypropylene (9) diglyceryl ether: SY-DP9 (manufactured by Sakamoto Yakuhin Kogyo Co., Ltd.)
*¹³polyoxybutylene polyoxyethylene polyoxypropylene glyceryl ether (3B.O.)(8E.O.)(5P.O.): WILBRIDE S-753 (manufactured by NOF Corp.)
*¹⁴polyethylene glycol (12) monolaurate: EMANON 1112 (manufactured by Kao Corp.)

### Examples 17 to 20 and Comparative Examples 11 to 15

Each skin cleansing composition was produced according to the composition shown in Table 4 and evaluated for the sustention of a smooth feel of the skin after rinsing, the sustention of the softness of the skin after rinsing, the feeling upon application after preservation, the fitting feeling of the solution upon massage, the moist feeling after rinsing, and the cleansing power against oil-based mascara. The results are also shown in Table 4.

### (Production Method)

Water was placed in a mixing vessel at room temperature. The component (B) was added thereto with stirring using Disper, and the mixture was stirred. If necessary, the component (E) was added thereto simultaneously with the component (B), and the mixture was stirred. Then, a base was added thereto, and the mixture was stirred. The component (A) and other components were further added thereto, and the mixture was stirred using a homomixer to produce a skin cleansing composition. In the case of adding a solid component at normal temperature, the component was thermally melted and then added to the mixing vessel, and the skin cleansing composition was produced in the same way as above, if necessary with heating, and then cooled to room temperature. In this production, the total amount of all components was 1 kg. The skin cleansing composition was produced such that the viscosity might be from 1,000 to 2,500 mPa·s.

### (Evaluation Method)

(1) Sustention of Smooth Feel of Skin after Rinsing:

Five expert panelists massaged their foundation-applied faces for approximately 5 seconds with both hands using 1 g of each cleansing composition and rinsed the faces five times using tap water. Then, the panelists kept quiet for 3 hours in a room of 30° C. and 60% RH and then conducted sensory evaluation for a smooth feel of the skin according to five levels given below. The sum of the scores of the five panelists was used for determination.

5: Smooth feel was sustained.
4: Smooth feel was slightly sustained.

### 3: Neutral.

2: Smooth feel was hardly sustained.
1: Smooth feel was not sustained at all.
(2) Sustention of Softness of Skin after Rinsing:

Five expert panelists massaged their foundation-applied faces for approximately 5 seconds with both hands using 1 g of each cleansing composition and rinsed the faces five times using tap water. Then, the panelists kept quiet for 3 hours in a room of 30° C. and 60% RH and then conducted sensory evaluation for the softness of the skin according to five levels given below. The sum of the scores of the five panelists was used for determination.

### 5: Soft.

4: Slightly soft.

### 3: Neutral.

2: Not very soft.
1: Not soft.
(3) Feeling Upon Application after Preservation (Fresh feeling):

Each skin cleansing composition was placed in a 100-mL glass bottle (diameter: approximately 5 cm, height: approximately 8 cm, manufacturer: TGK Co., Ltd.) and preserved at 50° C. for 4 weeks. Then, approximately 1 g was gently sucked out of each of the upper portion (between the surface of the solution and 5 mm below) and lower portion (between the bottom and 5 mm above) of the composition using a dropping syringe (material: polyethylene, volume: 2 mL). Five expert panelists massaged their faces for 5 seconds with both hands using these samples of the composition and rinsed the faces five times using tap water, followed by towel drying. Then, the panelists compared a fresh feeling and conducted sensory evaluation for difference in a feeling upon application according to five levels given below. The sum of the scores of the five panelists was used for determination.

### 5: Same.

4: Almost same.

### 3: Neutral.

2: Slightly different.

### 1: Different.

### (4) Fitting Feeling of Solution Upon Massage:

Five expert panelists massaged their foundation-applied faces for 5 seconds with both hands using 1 g of each cleansing composition. The panelists conducted sensory evaluation for a fitting feeling of the solution to the skin upon massage according to five levels given below. The sum of the scores of the five panelists was used for determination.

5: Fitting feeling was perceived.
4: Fitting feeling was slightly perceived.

### 3: Neutral.

2: Fitting feeling was hardly perceived.
1: Fitting feeling was not perceived at all.
(5) Moist Feeling after Rinsing:

Five expert panelists massaged their foundation-applied faces for 5 seconds with both hands using 1 g of each cleansing composition and rinsed the faces five times using tap water. Then, the panelists conducted sensory evaluation for a moist feeling according to five levels given below. The sum of the scores of the five panelists was used for determination.

### 5: Moist.

4: Slightly moist.

### 3: Neutral.

2: Not so moist.
1: Not moist.

### (6) Cleansing Power Against Oil-Based Mascara:

0.005 g of KOSE Sports Beauty Fasio Power Stay Mascara (Curl Long) BK001 (trade name) was uniformly applied as oil-based mascara (waterproof mascara) in a circular pattern of 1.2 cm in diameter onto a polypropylene plate (manufactured by Engineering Test Service (ETS)) and left for 2 hours for drying. Five expert panelists lightly rubbed the oil-based mascara for 10 seconds by finger massage using approximately 0.05 g of each cleansing composition and then rinsed off the composition in running water. The panelists evaluated the removal of the mascara according to five levels given below. The sum of the scores of the five panelists was used for determination.

5: Removed very well.
4: Removed well.

### 3: Neutral.

2: Not removed very well.
1: Not removed well.

TABLE 4

| | | Example | | | | Comparative Example | | | | |
|---|---|---|---|---|---|---|---|---|---|---|
| Component (mass %) | | 17 | 18 | 19 | 20 | 11 | 12 | 13 | 14 | 15 |
| A | Dioctyl ether*¹ | 14 | 2 | 2 | 2 | 0 | 2 | 0 | 14 | 0 |
| B | Hydroxyethylcellulose 1*⁴ | 5 | 5 | 5 | 2 | 0 | 0 | 2 | 0 | 2 |
| C | Water | 70.5549 | 71.485 | 71.485 | 74.485 | 76.395 | 76.485 | 74.485 | 76.485 | 88.485 |
| D | Isododecane*² (3 mPa · s) | 1 | 10 | 12 | 12 | 14 | 12 | 14 | 0 | 0 |
| | Isohexadecane*⁸ (5 mPa · s) | 0 | 1 | 0 | 0 | 0 | 0 | 0 | 0 | 0 |
| | Hydrogenated polyisobutene*⁹ | 0 | 1 | 0 | 0 | 0 | 0 | 0 | 0 | 0 |
| | (14 mPa · s) | | | | | | | | | |
| E | Acrylic acid/alkyl methacrylate | 0.0001 | 0.05 | 0.05 | 0.05 | 0.047 | 0.05 | 0.05 | 0.05 | 0.05 |
| | copolymer*¹⁰ | | | | | | | | | |
| | Sodium hydroxyethylcellulose | 0 | 0 | 0 | 0 | 0.093 | 0 | 0 | 0 | 0 |
| | hydroxypropyl stearyl ether | | | | | | | | | |
| | hydroxypropylsulfonate*¹¹ | | | | | | | | | |
| F | Polyoxypropylene (9) diglyceryl | 4 | 3 | 4 | 4 | 4 | 4 | 4 | 4 | 4 |
| | ether*¹² | | | | | | | | | |
| | Polyoxybutylene polyoxyethylene | 0 | 1 | 0 | 0 | 0 | 0 | 0 | 0 | 0 |
| | polyoxypropylene glyceryl ether | | | | | | | | | |
| | (3B.O.)(8E.O.)(5P.O.)*¹³ | | | | | | | | | |
| | 1,3-Butylene glycol | 5 | 5 | 5 | 5 | 5 | 5 | 5 | 5 | 5 |
| Others | Polyethylene glycol (12) | 0.045 | 0.045 | 0.045 | 0.045 | 0.045 | 0.045 | 0.045 | 0.045 | 0.045 |
| | monolaurate*¹⁴ | | | | | | | | | |
| | (HLB = 14) | | | | | | | | | |
| | Potassium hydroxide solution | 0.00004 | 0.02 | 0.02 | 0.02 | 0.02 | 0.02 | 0.02 | 0.02 | 0.02 |
| | (48%) | | | | | | | | | |
| | Methyl p-hydroxybenzoate | 0.1 | 0.1 | 0.1 | 0.1 | 0.1 | 0.1 | 0.1 | 0.1 | 0.1 |
| | Phenoxyethanol | 0.3 | 0.3 | 0.3 | 0.3 | 0.3 | 0.3 | 0.3 | 0.3 | 0.3 |
| Total | | 100 | 100 | 100 | 100 | 100 | 100 | 100 | 100 | 100 |
| B/A | | 0.36 | 2.50 | 2.50 | 1.00 | — | 0.00 | — | 0.00 | — |
| Ratio of A to whole oil phase | | 0.93 | 0.14 | 0.14 | 0.14 | 0.000 | 0.14 | 0.000 | 1.00 | — |
| A/C | | 0.20 | 0.03 | 0.03 | 0.03 | 0.00 | 0.03 | 0.00 | 0.18 | 0.00 |
| A + D | | 15.00 | 14.00 | 14.00 | 14.00 | 14.00 | 14.00 | 14.00 | 14.00 | 0.00 |
| A/((A) + (D)) | | 0.93 | 0.14 | 0.14 | 0.14 | 0.00 | 0.14 | 0.00 | 1.00 | — |
| Evalua- | Sustention of smooth feel of | 21 | 23 | 23 | 25 | 13 | 12 | 11 | 12 | 11 |
| tion | skin after rinsing | | | | | | | | | |
| | Sustention of softness of skin | 21 | 23 | 24 | 25 | 11 | 12 | 11 | 12 | 11 |
| | after rinsing | | | | | | | | | |
| | Feeling upon application after | 16 | 25 | 25 | 25 | 11 | 12 | 11 | 12 | 12 |
| | preservation(fresh feeling) | | | | | | | | | |
| | Fitting feeling of solution | 20 | 23 | 24 | 25 | 12 | 12 | 18 | 12 | 18 |
| | upon massage | | | | | | | | | |
| | Moist feeling after rinsing | 23 | 25 | 25 | 25 | 12 | 15 | 13 | 19 | 13 |
| | Cleansing power against | 21 | 20 | 21 | 24 | 17 | 24 | 17 | 24 | 5 |
| | oil-based mascara | | | | | | | | | |

*¹dioctyl ether: CETIOL OE (manufactured by BASF Japan Ltd.)
*²isododecane: MARUKASOL R (manufactured by Maruzen Petrochemical Co., Ltd.)
*⁴hydroxyethylcellulose 1: HEC Daicel SE400 (manufactured by Daicel FineChem Ltd., molecular weight (Mw): 250,000)
*⁸isohexadecane: PARLEAM 4 (manufactured by NOF Corp.)
*⁹hydrogenated polyisobutene: PARLEAM EX (manufactured by NOF Corp.)
*¹⁰acrylic acid/alkyl methacrylate copolymer: PEMULEN TR-2 (manufactured by Lubrizol Advanced Materials, Inc.)
*¹¹sodium hydroxyethylcellulose hydroxypropyl stearyl ether hydroxypropylsulfonate: SPS-S-SA (manufactured by Kao Corp.)
*¹²polyoxypropylene (9) diglyceryl ether: SY-DP9 (manufactured by Sakamoto Yakuhin Kogyo Co., Ltd.)
*¹³polyoxybutylene polyoxyethylene polyoxypropylene glyceryl ether (3B.O.)(8E.O.)(5P.O.): WILBRIDE S-753 (manufactured by NOF Corp.)
*¹⁴polyethylene glycol (12) monolaurate: EMANON 1112 (manufactured by Kao Corp.)

### Examples 21 to 25

In the same way as in Examples 4 to 16, each skin cleansing composition was produced according to the composition shown in Table 5 and evaluated for the sustention of a smooth feel of the skin after wiping, the sustention of the softness of skin after wiping, the sleekness of the skin after wiping, the fresh feeling after wiping, the moist feeling after wiping, and the cleansing power against oil-based mascara. The results are also shown in Table 5.

TABLE 5

| | | Example | | | | | |
|---|---|---|---|---|---|---|---|
| Component (mass %) | | 16 | 21 | 22 | 23 | 24 | 25 |
| A | Dioctyl ether*¹ | 2 | 2 | 2 | 2 | 2 | 2 |
| B | Hydroxyethylcellulose 1*⁴ | 0.1 | 0.1 | 0.1 | 0.1 | 0.1 | 0.1 |
| C | Water | 76.348 | 75.593 | 64.348 | 83.348 | 84.348 | 71.348 |
| D | Isododecane*² (3 mPa · s) | 12 | 12 | 12 | 5 | 12 | 12 |
| | Isohexadecane*⁸ (5 mPa · s) | 0 | 0 | 12 | 0 | 0 | 0 |
| E | Acrylic acid/alkyl methacrylate | 0.075 | 0.075 | 0.075 | 0.075 | 0.075 | 0.075 |
| | copolymer*¹⁰ | | | | | | |
| F | Polyoxypropylene (9) diglyceryl | 4 | 4 | 4 | 4 | 1 | 4 |
| | ether*¹² | | | | | | |
| | 1,3-Butylene glycol | 5 | 5 | 5 | 5 | 0 | 5 |
| | Glycerin | 0 | 0 | 0 | 0 | 0 | 2 |
| | Sorbitol | 0 | 0 | 0 | 0 | 0 | 3 |
| Others | Polyethylene glycol (12) | 0.045 | 0.8 | 0.045 | 0.045 | 0.045 | 0.045 |
| | monolaurate*¹⁴ | | | | | | |
| | (HLB = 14) | | | | | | |
| | Potassium hydroxide solution | 0.032 | 0.032 | 0.032 | 0.032 | 0.032 | 0.032 |
| | (48%) | | | | | | |
| | Methyl p-hydroxybenzoate | 0.1 | 0.1 | 0.1 | 0.1 | 0.1 | 0.1 |
| | Phenoxyethanol | 0.3 | 0.3 | 0.3 | 0.3 | 0.3 | 0.3 |
| Total | | 100.0 | 100.0 | 100.0 | 100.0 | 100.0 | 100.0 |
| B/A | | 0.05 | 0.05 | 0.05 | 0.05 | 0.05 | 0.05 |
| Ratio of A to whole oil phase | | 0.14 | 0.14 | 0.08 | 0.29 | 0.14 | 0.14 |
| A/C | | 0.03 | 0.03 | 0.03 | 0.02 | 0.02 | 0.03 |
| A + D | | 14.0 | 14.0 | 26.0 | 7.0 | 14.0 | 14.0 |
| A/((A) + (D)) | | 0.14 | 0.14 | 0.08 | 0.29 | 0.14 | 0.14 |
| Nonionic surfactant/total amount of oil agents | | 0.0032 | 0.057 | 0.0017 | 0.0064 | 0.0032 | 0.0032 |
| Evalua- | Sustention of smooth feel of | 25 | 24 | 24 | 25 | 25 | 24 |
| tion | skin after wiping | | | | | | |
| | Sustention of softness of skin | 25 | 24 | 25 | 25 | 25 | 24 |
| | after wiping | | | | | | |
| | Sleekness of skin after wiping | 24 | 24 | 25 | 24 | 24 | 25 |
| | Fresh feeling after wiping | 25 | 23 | 24 | 25 | 25 | 23 |
| | Moist feeling after wiping | 25 | 25 | 23 | 25 | 23 | 25 |
| | Cleansing power against | 25 | 23 | 25 | 24 | 25 | 25 |
| | oil-based mascara | | | | | | |

*¹dioctyl ether: CETIOL OE (manufactured by BASF Japan Ltd.)
*²isododecane: MARUKASOL R (manufactured by Maruzen Petrochemical Co., Ltd.)
*⁴hydroxyethylcellulose 1: HEC Daicel SE400 (manufactured by Daicel FineChem Ltd., molecular weight (Mw): 250,000)
*⁸isohexadecane: PARLEAM 4 (manufactured by NOF Corp.)
*¹⁰acrylic acid/alkyl methacrylate copolymer: PEMULEN TR-2 (manufactured by Lubrizol Advanced Materials, Inc.)
*¹²polyoxypropylene (9) diglyceryl ether SY-DP9 (manufactured by Sakamoto Yakuhin Kogyo Co., Ltd.)
*¹⁴polyethylene glycol (12) monolaurate: EMANON 1112 (manufactured by Kao Corp.)

1: A skin cleansing composition comprising components (A), (B), and (C):
(A) from 0.5 to 40 mass % of an ether oil in a liquid state at 25° C.,
(B) from 0.01 to 5 mass % of a nonionic water-soluble polymer comprising cellulose as a constitutional unit selected from the group consisting of methylcellulose, ethylcellulose, hydroxyethylcellulose, hydroxypropylcellulose, and hydroxypropylmethylcellulose, and
(C) water, and
1 mass % or less of a nonionic surfactant, wherein
the mass ratio of the component (B) to the component (A), (B)/(A) is from 0.001 to 5.

2: The skin cleansing composition according to claim 1, wherein the mass ratio of the component (A) to the whole oil phase, ((A)/whole oil phase amount)) is 0.05 or more.

3: The skin cleansing composition according to claim 1, wherein the component (A) is a dialkyl ether having two alkyl groups each having 16 or less of carbon atom.

4: The skin cleansing composition according to claim 1, wherein the component (A) is a dialkyl ether having two alkyl groups each having 8 or less of carbon atom.

5: The skin cleansing composition according to claim 1, wherein the component (B) is hydroxyethylcellulose.

6: The skin cleansing composition according to claim 1, further comprising (D) a hydrocarbon oil having a viscosity of 30 mPa·s or lower at 30° C.

7: The skin cleansing composition according to claim 1, further comprising (E) a water-soluble polymer comprising acrylic acid or methacrylic acid as a constitutional unit.

8: The skin cleansing composition according to claim 1, further comprising (F) a water-soluble solvent.

9: The skin cleansing composition according to claim 1, wherein the mass ratio of component (A) to component (C), (A)/(C) is from 0.005 to 1.

10: The skin cleansing composition according to claim 6, wherein the mass ratio between component (A) and component (D), (A)/((A)+(D)) is from 0.05 to 0.8.

11: The skin cleansing composition according to claim 6, wherein the content of component (D) in the total composition is from 1 mass % to 15 mass %.

12: The skin cleansing composition according to claim 6, wherein the component (D) is selected from the group consisting of isododecane, isohexadecane, and hydrogenated polyisobutene.

13: The skin cleansing composition according to claim 7, wherein the content of the component (E) in the total composition is from 0.0001 mass % to 0.3 mass %.

14: The skin cleansing composition according to claim 7, wherein the component (E) is an acrylic acid/alkyl methacrylate copolymer.

15: A method of cleansing a makeup cosmetic present on a face, comprising contacting the face have the makeup cosmetic present thereon with a skin cleansing composition according to claim 1.

16: A method of cleansing a makeup cosmetic present on a face, comprising contacting the face have the makeup cosmetic present thereon with a sheet having a skin cleansing composition according to claim 1 comprised therein.
